SEQUENCE LISTING (25 sequences)

SEQ ID NO: 1 - protein, 33 aa, Homo sapiens
SVSEIQLMHNLGKHLNSMERVEWLRKKLQDVHN

SEQ ID NO: 2 - protein, 31 aa, Homo sapiens
SVSEIQLMHNLGKHLNSMERVEWLRKKLQDV

SEQ ID NO: 3 - protein, 26 aa, Kaposi′s sarcoma-associated herpesvirus
AAVALLPAVLLALLAPVNRKRNKLMP

SEQ ID NO: 4 - protein, 11 aa, Human immunodeficiency virus
YGRKKRRQRRR

SEQ ID NO: 5 - protein, 15 aa, Homo sapiens
VTVLALGALAGVGVG

SEQ ID NO: 6 - protein, 12 aa, Kaposi′s sarcoma-associated herpesvirus
AAVLLPVLLAAP

SEQ ID NO: 7 - protein, 5 aa, Unknown
  Target fragment of Caspase-1
YVADX

SEQ ID NO: 8 - protein, 5 aa, Unknown
  Target fragment of Caspase-3
DXXDX

SEQ ID NO: 9 - protein, 5 aa, Unknown
  Target fragment of proprotein convertase 1
RXXRX

SEQ ID NO: 10 - protein, 5 aa, Unknown
  Target fragment of proprotein convertase 1
KXXRX

SEQ ID NO: 11 - protein, 6 aa, Unknown
  Target fragment of proprotein convertase 4
GRTKRX

SEQ ID NO: 12 - protein, 5 aa, Unknown
  Target fragment of proprotein convertase 4 PACE 4
RVRRX

SEQ ID NO: 13 - protein, 5 aa, Unknown
  Target fragment of proprotein convertase 4 PACE 4
RVRRX

SEQ ID NO: 14 - protein, 4 aa, Unknown
  Target fragment of Endothelin cleaving enzyme followed by dipeptidyl-peptidase IV
WVPX

SEQ ID NO: 15 - protein, 4 aa, Unknown
  Target fragment of Endothelin cleaving enzyme followed by dipeptidyl-peptidase IV
WVAX

SEQ ID NO: 16 - protein, 4 aa, Unknown
  Target fragment of Neprilysin followed by dipeptidyl-peptidase IV
XFXX

SEQ ID NO: 17 - protein, 4 aa, Unknown
  Target fragment of Neprilysin followed by dipeptidyl-peptidase IV
XYXX

SEQ ID NO: 18 - protein, 4 aa, Unknown
  Target fragment of Neprilysin followed by dipeptidyl-peptidase IV
XWXX

SEQ ID NO: 19 - protein, 12 aa, Unknown
  Target fragment of Renin followed by dipeptidyl-peptidase IV
DRYIPFHLLVYS

SEQ ID NO: 20 - protein, 32 aa, Homo sapiens
SVSEIQLMHNLGKHLNSMERVEWLRKKLQDVH

SEQ ID NO: 21 - protein, 33 aa, Homo sapiens; identical to SEQ ID NO: 1

SEQ ID NO: 22 - protein, 7 aa, Unknown
  Target fragment of proprotein convertase 1
RXXXXRX

SEQ ID NO: 23 - protein, 9 aa, Unknown
  Target fragment of proprotein convertase 1
RXXXXXXRX

SEQ ID NO: 24 - protein, 7 aa, Unknown
  Target fragment of proprotein convertase 1
KXXXXRX

SEQ ID NO: 25 - protein, 9 aa, Unknown
  Target fragment of proprotein convertase 1
KXXXXXXRX

(19) United States
(12) Patent Application Publication
(10) Pub. No.: US 2006/0270603 A1
(43) Pub. Date: Nov. 30, 2006

(54) Amidated parathyroid hormone fragments and uses thereof

(75) Inventors: Nozer M. Mehta, Randolph, NJ (US); William Stern, Tenafly, NJ (US); James P. Gilligan, Union, NJ (US); George B. Stroup, Malvern, PA (US)

Correspondence Address: OSTROLENK FABER GERB & SOFFEN, 1180 AVENUE OF THE AMERICAS, NEW YORK, NY, 100368403, US

(73) Assignees: Unigene Laboratories Inc.; Smithkline Beecham Corporation

(21) Appl. No.: 11/455,628

(22) Filed: Jun. 19, 2006

Related U.S. Application Data
(62) Division of application No. 11/040,557, filed on Jan. 21, 2005.
(60) Provisional application No. 60/538,403, filed on Jan. 21, 2004.

(51) Int. Cl. C07K 14/635

(52) U.S. Cl. 514/12; 530/399

(57) ABSTRACT

C-terminal amidated human parathyroid hormone analogs PTH 1-32-NH₂ and PTH 1-33-NH₂ are biologically active and can be used for the treatment of various bone related diseases and conditions.

### CROSS REFERENCE TO A RELATED APPLICATAIONS

The present application is a divisional of U.S. patent application Ser. No. 11/040,557, filed Jan. 21, 2005, which is a 35 U.S.C. §119 conversion of U.S. Provisional Application No. 60/538,403, filed Jan. 21, 2004.

### BACKGROUND OF THE INVENTION

1. Field of the Invention

The present invention relates to specific amidated fragments of parathyroid hormones that are biologically active, to pharmaceutical compositions, preferably oral compositions containing the same and to methods of using these fragments in treating osteoporosis and healing bone fracture.

2. Description of the Related Art

Numerous human hormones, neurotransmitters, cytokines, growth factors and other important biological compounds have peptides as a substantial part of their molecular structures. Many diseases respond positively to raising the level of these peptide compounds in patients. Therapeutically effective amounts of such biologically relevant peptides may be administered to patients in a variety of ways. However, as discussed further below, preferred oral administration is very difficult with this type of active compound.

Parathyroid hormone(PTH) is a peptide hormone produced by the parathyroid gland and is a major regulator of blood calcium levels. PTH is a polypeptide and synthetic polypeptides may be prepared by the method disclosed by Erickson and Merrifield, “Solid Phase Peptide Synthesis”, The Proteins, Third Edition, Neurath et al, Eds., Academic Press, New York, Vol. II, Chapter 3, pp. 255-257, 1976, and as modified by the method of Hodges, R. S. et al. “Protein Design Using Model Synthetic Peptides”, Peptide Research, Vol. 1, No. 1, pp. 19-30 (1998), or by Atherton, E. and Sheppard, R. C., “Solid Phase Peptide Synthesis—The Merrifield Technique”, Solid Phase Peptide Synthesis A Practical Approach, Chapter 2, pp. 13-23, IRL Press, Oxford University Press (1989).

When serum calcium is reduced to below a normal level, the parathyroid gland releases PTH and the calcium level is increased by resorption of bone calcium, by increased absorption of calcium from the intestine, and by increased renal reabsorption of calcium from nascent urine in the kidney tubules. Although continuously infused low levels of PTH can remove calcium from the bone, the same low doses, when intermittently injected can actually promote bone growth. This is because PTH given for a sufficient period of time, whether continuously infused or given intermittently by s.c. (subcutaneous) injection, will cause both a release of calcium from bone and an increase in bone formation. The difference is that the former is more likely to result in an unfavorable balance between bone resorption and formation leading to neutral or negative changes in bone mass, and to cause other negative effects such as marrow fibrosis, whereas the latter is likely to result in a positive balance in bone turnover, leading to increased bone mass.

Tregear, U.S. Pat. No. 4,086,196, described human PTH analogues and claimed that the first 27 to 34 amino acids are the most effective in terms of the stimulation of adenylyl cyclase in an in vitro cell assay. Rosenblatt, U.S. Pat. No. 4,771,124, disclosed the property of hPTH analogues wherein Trp²³ is substituted by amino acids phenylalanine, leucine, norleucine, valine, tyrosine, b-naphthylalanine, or a-naphthylalanine as a PTH antagonist. These modified hPTH analogues also have the 2 and 6 amino terminal acids removed, resulting in loss of most agonist activities when used to treat osteoporosis. These analogues were designed as inhibitors of PTH and PTH-related peptides. The analogues were claimed as possibly useful in the treatment of hypercalcemia associated with some tumors.

Pang et al, WO93/06845, published Apr. 15, 1993, described analogues of hPTH which involve substitutions of Arg²⁵, Lys²⁶, Lys²⁷ with numerous amino acids, including alanine, asparagine, aspartic acid, cysteine, glutamine, glutamic acid, glycine, histidine, isoleucine, leucine, methionine, phenylalanine, proline, serine, threonine, tryptophan, tyrosine, or valine. These are claimed to be effective in the treatment of osteoporosis with minimal effects on blood pressure and smooth muscle.

PTH operates through activation of two second messenger systems, Gₛ-protein activated adenylyl cyclase (AC) and G_{q}-protein activated phospholipase C_{b}. The latter results in a stimulation of membrane-bound protein kinase Cs (PKC) activity. The PKC activity has been shown to require PTH residues 29 to 32 (Jouishomme et al (1994) J. Bone Mineral Res. 9, pp. 943-949. It is believed that the increase in bone growth, i.e., that effect which is useful in the treatment of osteoporosis, is coupled to the ability of the peptide sequence to increase AC activity. The native PTH sequence has been shown to have all of these activities. The truncated human hPTH-(1-34) sequence is typically shown as:
Ser Val Ser Glu Ile Gln Leu Met His Asn Leu Gly Lys His Leu Asn Ser Met Glu Arg Val Glu Trp Leu Arg Lys Lys Leu Gln Asp Val His Asn Phe-OH (SEQ ID NO:1).

Various PTH analogs are disclosed in U.S. Pat. Nos. 5,955,425 and 6,110,892. The following linear analogue (truncated hPTH), hPTH-(1-31)-NH₂, has only AC-stimulating activity and has been shown to be fully active in the restoration of bone loss in the ovariectomized rat model (Rixon, R. H. et al (1994) J. Bone Miner. Res. 9, 1179-1189; Whitfield et al (1996), Calcified Tissue Int. 58, 81-87; and Willick et al, U.S. Pat. No. 5,556,940):
Ser Val Ser Glu Ile Gln Leu Met His Asn Leu Gly Lys His Leu Asn Ser Met Glu Arg Val Glu Trp Leu Arg Lys Lys Leu Gln Asp Val-NH₂ (SEQ ID NO:2).

U.S. patent application Ser. No. 10/761,481 filed Jan. 20, 2004, claiming priority of 60/441,856, filed Jan. 21, 2003 entitled “Improved Oral delivery of peptides” by inventors Nozer M. Mehta, William Stern and James Gilligan discloses that the amidation of peptide hormones such as parathyroid hormone fragments enhances their bioavailability when administered orally. However, this application does not measure the biological activities of the various PTH fragments.

Peptide pharmaceuticals used in the prior art frequently have been administered by injection or by nasal administration. Insulin is one example of a peptide pharmaceutical frequently administered by injection. A more preferred and convenient oral administration tends to be problematic because peptide active compounds are very susceptible to degradation in the stomach and intestines. For example, while the prior art has reported an ability to achieve reproducible blood levels of salmon calcitonin and parathyroid hormone when administered orally, these levels are low. This is believed to be because these peptide hormones lack sufficient stability in the gastrointestinal tract, and tend to be poorly transported through intestinal walls into the blood. However, injection and nasal administration are significantly less convenient than, and involve more patient discomfort than, oral administration. Often this inconvenience or discomfort results in substantial patient noncompliance with a treatment regimen. Thus, there is a need in the art for more effective and reproducible oral administration of peptide pharmaceuticals like insulin, salmon calcitonin, parathyroid hormone and others discussed in more detail herein.

Proteolytic enzymes of both the stomach and intestines may degrade peptides, rendering them inactive before they can be absorbed into the bloodstream. Any amount of peptide that survives proteolytic degradation by proteases of the stomach (typically having acidic pH optima) is later confronted with proteases of the small intestine and enzymes secreted by the pancreas (typically having neutral to basic pH optima). Specific difficulties arising from the oral administration of a peptide like salmon calcitonin involve the relatively large size of the molecule, and the charge distribution it carries. This may make it more difficult for salmon calcitonin to penetrate the mucus along intestinal walls or to cross the intestinal brush border membrane into the blood.

One way to improve the effectiveness of oral administration of peptides is to protect them from proteolytic enzymes in the stomach and intestine as well as enhance their absorption from the intestine thereby enhancing their bioavailability. Improving oral effectiveness is important for several reasons. First, peptides and proteins are expensive to manufacture either by chemical synthesis or recombinant DNA technologies. Therefore, the more one increases bioavailability, the lesser the amounts that will be required in an oral formulation of a therapeutic drug.

Second, the greater the bioavailability of an oral peptide, the less the variability in the dosage absorbed by an individual on a day to day basis.

Third, the greater the bioavailability of an oral peptide, the less the concern about breakdown products of the peptide since such breakdown products can act as agonists or antagonists of the receptors where the peptide binds to elicit biological activity.

### SUMMARY OF THE INVENTION

It is accordingly an object of the present invention to provide C-terminal amidated human parathyroid hormone analogs PTH 1-32-NH₂ and PTH 1-33-NH₂.

The human hPTH-(1-32) sequence is as follows:

(SEQ ID NO:20)

Ser Val Ser Glu Ile Gln Leu Met His Asn Leu Gly
Lys His Leu Asn Ser Met Glu Arg Val Glu Trp Leu
Arg Lys Lys Leu Gln Asp Val His.

The human hPTH-(1-33) sequence is typically shown as:

(SEQ ID NO:21)

Ser Val Ser Glu Ile Gln Leu Met His Asn Leu Gly
Lys His Leu Asn Ser Met Glu Arg Val Glu Trp Leu
Arg Lys Lys Leu Gln Asp Val His Asn

It is a further object of the invention to provide a therapeutically effective pharmaceutical composition for delivering C-terminal amidated human parathyroid hormone analogs PTH 1-32-NH₂ and PTH 1-33-NH₂.

It is a further object of the invention to provide methods of treating or preventing bone-related diseases such as osteoporosis, calcium disorders and methods for accelerating the healing of a broken bone by administering a C-terminal amidated human parathyroid hormone analog PTH 1-32-NH₂ or PTH 1-33-NH₂.

It is another object of the invention to provide agents that are easily manufactured, have enhanced bioavailability, good shelf stability and reduce the undesired side effects associated with the use of ful-length parathyroid hormone such as hypercalcemia.

In one aspect, the invention provides a pharmaceutical composition for oral delivery of a C-terminal amidated human parathyroid hormone analog PTH 1-32-NH₂ or PTH 1-33-NH₂ comprising a therapeutically effective amount of said analog.

The present invention is believed to reduce the likelihood of proteolytic degradation of the peptide active compound by simultaneously protecting the peptide from proteolytic attack by (1) stomach proteases which are typically most active at acidic pHs and (2) intestinal or pancreatic proteases (which are typically most active at basic to neutral pH).

Also, the invention is believed to promote the process by which the peptide crosses the intestinal brush border membrane into the blood due to the presence of amide, while continuing to protect the peptide from proteolytic degradation.

An acid resistant protective coating of the capsule or tablet protects the PTH analog from the acid-acting proteases of the stomach. Thereafter, after the formulation passes into the intestine where the pH is less acidic, the enteric coating dissolves to release the contents of the formulation. Significant quantities of acid (with which the peptide active agent is intermixed) reduce the activity of neutral to basic-acting proteases (e.g., luminal or digestive proteases and proteases of the brush border membrane) by lowering pH locally at the site of release of the formulation below their optimal activity range.

A patient in need of treatment or reducing the risk of onset of a given disease is one who has either been diagnosed with such disease or one who is susceptible to acquiring such disease. The invention is especially useful for individuals who, due to heredity, environmental factors or other recognized risk factor, are at higher risk than the general population of acquiring the conditions to which the present invention relates.

Except where otherwise stated, the preferred dosage for each active component discussed herein is the same regardless of the disease being treated (or prevented).

Except where otherwise noted or where apparent from context, dosages herein refer to weight of active compounds unaffected by pharmaceutical excipients, diluents, carries or other ingredients, although such additional ingredients are desirably included, as discussed elsewhere herein. Any dosage form (capsule, tablet, injection or the like) commonly used in the pharmaceutical industry is appropriate for use herein, and the terms “excipient,” “diluent” or “carrier” include such non-active ingredients as are typically included, together with active ingredients in such dosage forms in the industry. For example, typical capsules, pills, enteric coatings, solid or liquid diluents or excipients, flavorants, preservatives, or the like are included.

Other features and advantages of the present invention will become apparent from the following detailed description of the invention.

### DETAILED DESCRIPTION OF THE INVENTION

In accordance with the invention, patients in need of treatment with parathyroid hormone are provided with a pharmaceutical composition containing a C-terminal amidated human parathyroid hormone analog PTH 1-32-NH₂ or PTH 1-33-NH₂ (at appropriate dosage), preferably but not necessarily in oral formulations such as tablet or capsule form of an ordinary size in the pharmaceutical industry.

Applicants have discovered that the C-terminal amidated human parathyroid hormone analogs PTH 1-32-NH₂ and PTH 1-33-NH₂ are biologically active. Patients who may benefit are any who suffer from disorders that respond favorably to increased levels of parathyroid hormone. The invention may be used, for example, to treat bone fracture, osteoporosis, Paget's disease, hypercalcemia of malignancy and the like.

Without intending to be bound by theory, the pharmaceutical composition of the invention, when prepared for oral administration, is expected to overcome a series of different and unrelated natural barriers to bioavailability. Various components of the oral pharmaceutical compositions are directed to overcome different barriers by mechanisms appropriate to each, and to result in synergistic effects on the bioavailability of a peptide active ingredient. Suitable oral delivery technology is taught, for example, in U.S. Pat. No. 6,086,918, the entire specification of which is hereby incorporated by reference.

It is also believed that the present human parathyroid hormone analogs PTH 1-32-NH₂ and PTH 1-33-NH₂ may be efficiently manufactured with recombinant direct expression as discussed hereinbelow, and amidated at the C-terminal site. In accordance with the invention, the presence of at least one amide group is believed to help protect the peptide or protein from proteolytic degradation, thereby improving bioavailability. The amide group may also enhance the membrane permeability of the protein across the lumen of the intestine. Other mechanisms for increase in bioavailability by the presence of the amide group may also be possible.

Various techniques exist for recombinant production of the present PTH analogs.

Overview of a Preferred Expression Vector

A preferred expression vector is described in U.S. Pat. No. 6,210,925 and is incorporated herein by reference. An example of a preferred vector for expressing salmon calcitonin is shown in FIG. 9 of U.S. Pat. No. 6,210,925. For the expression of the present PTH analogs, nucleic acids coding for the analog would be substituted for the nucleic acid coding for salmon calcitonin.

The preferred expression vector comprises a coding region and a control region. The coding region comprises nucleic acids for PTH analog PTH 1-32 or PTH 1-33 coupled in reading frame downstream from nucleic acids coding for a signal peptide. The control region is linked operably to the coding region and comprises a plurality of promoters and at least one ribosome binding site, wherein at least one of the promoters is selected from the group consisting of tac and lac.

Preferably, the vector comprises a plurality of transcription cassettes placed in tandem, each cassette having the control region and the coding region of the present invention. Such a digenic vector or multigenic vector is believed to provide better expression than would a dicistronic or multicistronic expression vector.

The vector can optionally further comprise nucleic acids coding for a repressor peptide which represses operators associated with one or more of the promoters in the control region, a transcription terminator region, a selectable marker region and/or a region encoding at least one secretion enhancing peptide. Alternatively, in some embodiments, nucleic acids coding for a repressor peptide and a secretion enhancing peptide may be present on a separate vector co-expressed in the same host cell as the vector expressing the peptide product.

Many commercially available vectors may be utilized as starting vectors for the preferred vectors of the invention. Some of the preferred regions of the vectors of the invention may already be included in the starting vector such that the number of modifications required to obtain the vector of the invention is relatively modest.

The Control Region

The control region is operably linked to the coding region and comprises a plurality of promoters and at least one ribosome binding site, wherein at least one of the promoters is selected from the group consisting of lac and tac. Other promoters are known in the art, and may be used in combination with a tac or lac promoter. Such promoters include but are not limited to lpp, ara B, trpE, gal K.

Preferably, the control region comprises exactly two promoters. When one of the promoters is tac, it is preferred that the tac promoter be 5′ of another promoter in the control region. When one of the promoters is lac, the lac promoter is preferably 3′ of another promoter in the control region. Also preferably, the control region comprises both a tac promoter and a lac promoter, preferably with the lac promoter being 3′ of the tac promoter.

The Coding Region

The coding region comprises nucleic acids coding for present glycine-extended PTH analog coupled in reading frame downstream from nucleic acids coding for a signal peptide whereby the coding region encodes a peptide comprising, respectively, from N terminus to C terminus the signal and the glycine-extended PTH analog. Without intending to be bound by theory, it is believed that the signal may provide some protection to the peptide product from proteolytic degradation in addition to participating in its secretion to the periplasm.

Many peptide signal sequences are known and may be used in accordance with the invention. These include signal sequences of outer membrane proteins of well-characterized host cells, and any sequences capable of translocating the peptide product to the periplasm and of being post-translationally cleaved by the host as a result of the translocation. Useful signal peptides include but are not limited to Omp A, pel B, Omp C, Omp F, Omp T, β-la, Pho A, Pho S and Staph A.

The glycine-extended PTH analog is used as a precursor to an enzymatic amidation reaction converting the C-terminal amino acid to an amino group, thus resulting in an amidated analog. Such a conversion of in a peptide of a C-terminal amino acid to an amino group is described in more detail infra.

Other Optional Aspects of a Preferred Vector of the Invention or of Other Vectors to be Expressed in the Same Host as the Vector of the Invention

Repressor

Optionally, the preferred vector may contain nucleic acids coding for a repressor peptide capable of repressing expression controlled by at least one of the promoters. Alternatively, however, the nucleic acids coding for a repressor peptide may be present on a separate vector in a host cell with the vector of the present invention. Appropriate repressors are known in the art for a large number of operators. Preferably, the nucleic acids coding for the repressor encode a lac repressor in preferred embodiments of the invention because it represses the lac operator that is included with both tac and lac promoters, at least one of which promoters is always present in preferred vectors of the invention.

Selectable Marker

It is preferred that any of a large number of selectable marker genes (e.g. a gene encoding kanamycin resistance) be present in the vector. This will permit appropriate specific selection of host cells that are effectively transformed or transfected with the novel vector of the invention.

Secretion Enhancing Peptide

Nucleic acids coding for at least one secretion enhancing peptide are optionally present in the vector of the present invention. Alternatively, the nucleic acids coding for a secretion enhancing peptide may be present on a separate vector expressed in the same host cell as the vector encoding the peptide product. Preferably, the secretion enhancing peptide is selected from the group consisting of SecY (prlA) or prlA-4. It is pointed out that SecY and prlA are identical, the two terms being used as synonyms in the art. prlA-4 is a known modification of prlA and has a similar function. Another preferred secretion enhancing peptide is SecE also known as “prlG”, a term used as a synonym for “SecE”. Most preferably, a plurality of secretion enhancing peptides are encoded, at least one of which is SecE and the other of which is selected from the group consisting of SecY (prlA) and prlA-4. The two are believed to interact to aid translocation of the peptide product from cytoplasm to periplasm. Without intending to be bound by theory, these secretion enhancing peptides may help protect the PTH analog from cytoplasmic proteases in addition to their secretion enhancing functions.

Amidation of peptides and proteins, preferably at the C-terminus is bellied to afford a significant increase in oral bioavailability.

Normally, the plasma membrane of eukaryotic cells is impermeable to large peptides or proteins. However, certain hydrophobic moieties such as amino acid sequences, fatty acids and bile acids variously called ferry peptides or membrane translocating sequences or moieties, when fused to the functional proteins or peptides, in particular to the N— or C-terminus, can act as membrane translocators, and mediate the transport of these proteins into living cells. These membrane translocators (MTs) for the purpose of the present invention are capable of being at least partially cleaved by a blood or lymphatic system protease. Suitable oral delivery technology using membrane translocators is taught, for example, in U.S. Pat. No. 6,673,574 the entire specification of which is hereby incorporated by reference.

In accordance with another aspect of the invention, the presence of at least one membrane translocator (MT), preferably two MTs, more preferably, two peptide MTs is used. This is expected to enhance the membrane permeability of the PTH analog fused to the MT(s) across the lumen of the intestine and provide for improved bioavailability. Since the MT link to the active peptide can be cleaved by an enzyme in the blood or the lymphatic system, it can leave the active peptide free to reach its target.

Also, in accordance with the invention, proteolytic degradation of the PTH analog and of the membrane translocator by stomach enzymes (most of which are active in the acid pH range) and intestinal or pancreatic proteases (most of which are active in the neutral to basic pH range) is reduced.

Again, without intending to be bound by theory, it is expected that, in accordance with the present invention, the PTH analog is transported through the stomach under the protection of an appropriate acid-resistant protective vehicle for substantially preventing contact between the salmon calcitonin or other active peptide and any stomach proteases capable of degrading it. Once the pharmaceutical composition of the invention passes through the stomach and enters the intestinal region where basic to neutral pH predominates, and where proteases tend to have basic to neutral pH optima, the enteric coating or other vehicle releases the PTH analog and acid or protease inhibitors (in close proximity to each other).

The acid is believed to lower the local intestinal pH (where the active agent PTH analog has been released) to levels below the optimal range for many intestinal proteases and other intestinal enzymes. This decrease in pH is believed to reduce the proteolytic activity of the intestinal proteases, thus affording protection to the PTH analog and the membrane translocator from potential degradation. The activity of these proteases is diminished by the temporarily acidic environment provided by the invention. It is preferred that sufficient acid be provided that local intestinal pH is lowered temporarily to 5.5 or below, preferably 4.7 or below and more preferably 3.5 or below. The sodium bicarbonate test described below (in the section captioned “the pH-Lowering Agent”) is indicative of the required acid amount. Preferably, conditions of reduced intestinal pH persist for a time period sufficient to protect the PTH analog and the membrane translocator from proteolytic degradation until at least some of the peptide agent has had an opportunity to cross the intestinal wall into the bloodstream.

Alternatively, or in addition, protease inhibitors are used and are believed to reduce the proteolytic activity of the intestinal proteases, thus affording protection to the PTH analog and the membrane translocator from premature potential degradation.

Compositions of the present invention can optionally contain absorption enhancers. The absorption enhancers of the invention synergistically promote peptide absorption into the blood while conditions of reduced proteolytic activity prevail.

The mechanism by which the invention is believed to accomplish the goal of enhanced bioavailability is aided by having active components of the pharmaceutical composition released together as simultaneously as possible. To this end, it is preferred to keep the volume of enteric coating as low as possible consistent with providing protection from stomach proteases. Thus enteric coating is less likely to interfere with PTH analog release, or with the release of other components in close time proximity with the peptide. The enteric coating should normally add less than 30% to the weight of the remainder of pharmaceutical composition (i.e., the other components of the composition excluding enteric coating). Preferably, it is less than 20% and, more preferably, the enteric coating adds between 10% and 20% to the weight of the uncoated ingredients.

The absorption enhancer which may be a solubility enhancer and/or transport enhancer (as described in more detail below) aids transport of the peptide agent from the intestine to the blood, and may promote the process so that it better occurs during the time period of reduced intestinal pH and reduced intestinal proteolytic activity. Many surface active agents may act as both solubility enhancers and transport (uptake) enhancers. Again without intending to be bound by theory, it is believed that enhancing solubility provides (1) a more simultaneous release of the active components of the invention into the aqueous portion of the intestine, (2) better solubility of the peptide in, and transport through, a mucous layer along the intestinal walls. Once the peptide active ingredient reaches the intestinal walls, an uptake enhancer is expected to provide better transport through the brush border membrane of the intestine into the blood, via either transcellular or paracellular transport. As discussed in more detail below, many preferred compounds may provide both functions. In those instances, preferred embodiments utilizing both of these functions may do so by adding only one additional compound to the pharmaceutical composition. In other embodiments, separate absorption enhancers may provide the two functions separately.

Each of the preferred ingredients of the pharmaceutical composition of the invention is separately discussed below. Combinations of multiple pH-lowering agents, or multiple enhancers can be used as well as using just a single pH-lowering agent and/or single enhancer. Some preferred combinations are also discussed below.

Peptide Active Ingredients

Amidation of the peptide can be achieved either by chemical or enzymatic means, or by a combination of the two. A preferred method of amidation is by the action of peptidylglycine-amidating monooxygenase on a substrate that a C-terminal glycine that is to become C-terminal —NH₂ in the desired product.

The PTH analog may be extended by a glycine at the C-terminal end when produced by recombinant technology and the C-terminus is amidated by enzymatic reaction. Alternatively, amino acid side chains suitable for amidation can also be amidated by chemical reaction.

Also, preferably, the PTH analog of the present invention is linked to an MT sequence to facilitate its absorption from the intestine. The MT must be protected from cleavage by proteases in the stomach and intestine before its absorption. However, once absorbed, the MT should be able to be at least partially removed by proteases to free up the active peptide.

The MT can comprise an amino acid sequence, preferably a signal peptide or signal sequence. A “signal peptide,” as used herein, is a sequence of amino acids generally but not necessarily of a length of about 10 to about 50 or more amino acid residues, many (typically about 55-60%) residues of which are hydrophobic such that they have a hydrophobic, lipid-soluble portion. The hydrophobic portion is a common, major motif of the signal peptide, and it is often a central part of the signal peptide of proteins secreted from cells. A signal peptide is a sequence of amino acids that facilitates the export of cytoplasmic proteins. The signal peptides of this invention, as discovered herein, are also “importation competent,” i.e., capable of penetrating through the cell membrane from outside the cell to the interior of the cell. The amino acid residues can be mutated and/or modified (i.e., to form mimetics) so long as the modifications do not affect the translocation-mediating function of the peptide. Thus the word “peptide” includes mimetics and the word “amino acid” includes modified amino acids, as used herein, unusual amino acids, and D-form amino acids. All importation competent signal peptides encompassed by this invention have the function of mediating translocation across a cell membrane from outside the cell to the interior of the cell. They may also retain their ability to allow the export of a protein from the cell into the external milieu. A putative signal peptide can easily be tested for this importation activity following the teachings provided herein, including testing for specificity for any selected cell type.

The following Table 1 exemplifies amino acid sequences, each of which can be used as an MT.

TABLE 1

Amino Acid Sequences of Some
MT Peptides and Their Sources

| | SEQUENCE | |
|---|---|---|
| SEQUENCE | DERIVATION | SOURCE |
| ALA-ALA-VAL-ALA-LEU- | Signal Peptide | U.S. Pat. 5,807,746 |
| LEU-PRO-ALA-VAL-LEU- | from Kaposi | |
| LEU-ALA-LEU-LEU-ALA- | Fibroblast Growth | |
| PRO-VAL-ASN-ARG-LYS- | Factor | |
| ARG-ASN-LYS-LEU-MET- | | |
| PRO (SEQ ID No:3) | | |
| TYR-GLY-ARG-LYS-LYS- | Protein | Schwarz et al. (1999), |
| ARG-ARG-GLN-ARG-ARG- | Transduction | Science 285:1569 |
| ARG (SEQ ID No:4) | Domain of HIV TAT | |
| | Protein | |
| VAL-THR-VAL-LEU-ALA- | Signal Sequence of | Zhang et al. (1988) |
| LEU-GLY-ALA-LEU-ALA- | Human Integrin β₃ | PNAS 95:9184 |
| GLY-VAL-GLY-VAL-GLY | | |
| (SEQ ID No:5) | | |
| 38 kDa Protein | HSV-VP22 Protein | Phelan et al. (1998), |
| | | Nature Biotechnology |
| | | 16:440 |
| ALA-ALA-VAL-LEU-LEU- | Modified from 16- | Rojas et al (1998) |
| PRO-VAL-LEU-LEU-ALA- | residue | Nature Biotechnology |
| ALA-PRO | hydrophobic region | 16:370 |
| (SEQ ID No:6) | of signal sequence | |
| | of Kaposi | |
| | fibroblast growth | |
| | factor | |

The MT can also comprise fatty acids and/or bile acids. Such molecules, when used, are linked to the present PTH analog by an amino acid bridge which is subject to cleavage by proteases in the plasma. Alternatively, the MT can be linked to the PTH analog by a non-peptidyl linkage, in which case the in vivo enzyme that cleaves the linkage may be an enzyme other than protease. The amino acid bridge must be a target for cleavage by at least one plasma protease. Plasma proteases as well as their target sequences are well known in the art. Table 2 illustrates some of these enzymes as well as their specific targets

TABLE 2

Plasma Proteases and their Specific Targets

| | | |
|---|---|---|
| PROTEASE | SPECIFIC TARGET | REMARKS |

| | | |
|---|---|---|
| Caspase-1 | Tyr-Val-Ala-Asp-Xaa* | |
| | (SEQ ID No:7) | |
| Caspase-3 | Asp-Xaa-Xaa-Asp-Xaa | |
| | (SEQ ID No:8) | |
| Proprotein | Arg-(Xaa)n-Arg-Xaa | n=2, 4 or 6 |
| convertase 1 | (SEQ ID Nos. 9, 22 | |
| | & 23) | |
| | Lys-(Xaa)n-Arg-Xaa | n=2, 4, or 6 |
| | (SEQ ID Nos. 10, 24 | |
| | & 25) | |
| | Arg-Arg-Xaa | |
| | Lys-Arg-Xaa | |
| Proprotein | same as proprotein | |
| convertase 2 | convertase 1 | |
| Proprotein | Gly-Arg-Thr-Lys-Arg- | |
| convertase 4 | Xaa (SEQ ID No:11) | |
| Proprotein | Arg-Val-Arg-Arg-Xaa | |
| convertase | (SEQ ID No:12) | |
| 4 PACE 4 | | |
| | Decanoyl-Arg-Val- | |
| | Arg-Arg-Xaa | |
| | (SEQ ID No: 13) | |
| Prolyl | Pro-Xaa | |
| oligopeptidase | | |
| Endothelin cleaving | Trp-Val-Pro-Xaa | |
| enzyme followed by | (SEQ ID No:14) | |
| dipeptidyl- | Trp-Val-Ala-Xaa | |
| peptidase IV | (SEQ ID No:15) | |
| Signal peptidase | | depends on nearby |
| | | amino acid |
| Neprilysin followed | Xaa-Phe-Xaa-Xaa | broad specificity, |
| by dipeptidyl- | (SEQ ID No:16) | max length = 40 amino |
| peptidase IV | | acids |
| | Xaa-Tyr-Xaa-Xaa | |
| | (SEQ ID No:17) | |
| | Xaa-Trp-Xaa-Xaa | |
| | (SEQ ID No:18) | |
| Renin followed by | Asp-Arg-Tyr-Ile-Pro- | substitute Pro or Ala |
| dipeptidyl-peptidase | Phe-His-Leu-Leu-Val- | for Val & Ser |
| IV | Tyr-Ser | |
| | (SEQ ID No: 19) | |

*The N-terminal side of bolded amino acids is the specific target for the protease cleavage.

The invention, by several mechanisms, suppresses the degradation of the active ingredient by protease that would otherwise tend to cleave one or more of the peptide bonds of the active ingredient.

When an MT is linked to the active PTH analog ingredient of the invention, it may be accomplished by either chemical or recombinant syntheses known in the art. By “linking” as used herein is meant that the biologically active peptide is associated with the MT in such a manner that when the MT crosses the cell membrane, the PTH analog is also imported across the cell membrane. Examples of such means of linking include (A) linking the MT to the PTH analog by a peptide bond, i.e., the two peptides (the peptide part of the MT and the active peptide PTH analog) can be synthesized contiguously; (B) linking the MT to the active peptide by a non-peptide covalent bond (such as conjugating a signal peptide to a protein with a crosslinking reagent); (C) chemical ligation methods can be employed to create a covalent bond between the carboxy-terminal amino acid of an MT such as a signal peptide and the active peptide.

Examples of method (A) are shown below wherein a peptide is synthesized, by standard means known in the art, (Merrifield, J. Am. Chem. Soc. 85:2149-2154, 1963; and Lin et al., Biochemistry 27:5640-5645, 1988) and contains, in linear order from the amino-terminal end, a signal peptide sequence (the MT), an amino acid sequence that can be cleaved by a plasma protease, and a biologically active amino acid sequence. Such a peptide could also be produced through recombinant DNA techniques, expressed from a recombinant construct encoding the above-described amino acids to create the peptide. (Sambrook et al., Molecular Cloning: A Laboratory Manual, 2nd Ed., Cold Spring Harbor Laboratory, Cold Spring Harbor, N.Y., 1989).

For method (B), either a peptide bond, as above, can be utilized or a non-peptide covalent bond can be used to link the MT with the biologically active peptide, polypeptide or protein. This non-peptide covalent bond can be formed by methods standard in the art, such as by conjugating the MT to the peptide, polypeptide or protein via a crosslinking reagent, for example, glutaraldehyde. Such methods are standard in the art. (Walter et al., Proc. Natl. Acad. Sci. USA 77:5197; 1980).

For method (C), standard chemical ligation methods, such as using chemical crosslinkers interacting with the carboxy-terminal amino acid of a signal peptide, can be utilized. Such methods are standard in the art (Goodfriend et al., Science 144:1344-1346, 1964, which uses water-soluble carbodiimide as a ligating reagent) and can readily be performed.

The pH-Lowering Agent and Protease Inhibitor

The total amount of the pH-lowering compound to be administered with each administration of PTH analog should preferably be an amount which, when it is released into the intestine, is sufficient to lower the local intestinal pH substantially below the pH optima for proteases found there. The quantity required will necessarily vary with several factors including the type of pH-lowering agent used (discussed below) and the equivalents of protons provided by a given pH-lowering agent. In practice, the amount required to provide good bioavailability is an amount which, when added to a solution of 10 milliliters of 0.1 M sodium bicarbonate, lowers the pH of that sodium bicarbonate solution to no higher than 5.5, and preferably no higher than 4.7, most preferably no higher than 3.5. Enough acid to lower pH, in the foregoing test, to about 2.8 may be used in some embodiments. Preferably at least 300 milligrams, and more preferably at least 400 milligrams of the pH-lowering agent are used in the pharmaceutical composition of the invention. The foregoing preferences relate to the total combined weight of all pH-lowering agents where two or more of such agents are used in combination. The oral formulation should not include an amount of any base which, when released together with the pH-lowering compound, would prevent the pH of the above-described sodium bicarbonate test from dropping to 5.5 or below.

The pH-lowering agent of the invention may be any pharmaceutically acceptable compound that is not toxic in the gastrointestinal tract and is capable of either delivering hydrogen ions (a traditional acid) or of inducing higher hydrogen ion content from the local environment. It may also be any combination of such compounds. It is preferred that at least one pH-lowering agent used in the invention have a pKa no higher than 4.2, and preferably no higher than 3.0. It is also preferred that the pH lowering agent have a solubility in water of at least 30 grams per 100 milliliters of water at room temperature.

Examples of compounds that induce higher hydrogen ion content include aluminum chloride and zinc chloride. Pharmaceutically acceptable traditional acids include, but are not limited to acid salts of amino acids (e.g., amino acid hydrochlorides) or derivatives thereof. Examples of these are acid salts of acetylglutamic acid, alanine, arginine, asparagine, aspartic acid, betaine, carnitine, carnosine, citrulline, creatine, glutamic acid, glycine, histidine, hydroxylysine, hydroxyproline, hypotaurine, isoleucine, leucine, lysine, methylhistidine, norleucine, ornithine, phenylalanine, proline, sarcosine, serine, taurine, threonine, tryptophan, tyrosine and valine.

Other examples of useful pH-lowering compounds include carboxylic acids such as acetylsalicylic, acetic, ascorbic, citric, fumaric, glucuronic, glutaric, glyceric, glycocolic, glyoxylic, isocitric, isovaleric, lactic, maleic, oxaloacetic, oxalosuccinic, propionic, pyruvic, succinic, tartaric, valeric, and the like.

Other useful pH-lowering agents that might not usually be called “acids” in the art, but which may nonetheless be useful in accordance with the invention are phosphate esters (e.g., fructose 1, 6 diphosphate, glucose 1, 6 diphosphate, phosphoglyceric acid, and diphosphoglyceric acid). CARBOPOL® (Trademark BF Goodrich) and polymers such as polycarbophil may also be used to lower pH.

Any combination of pH lowering agent that achieves the required pH level of no higher than 5.5 in the sodium bicarbonate test discussed above may be used. One preferred embodiment utilizes, as at least one of the pH-lowering agents of the pharmaceutical composition, an acid selected from the group consisting of citric acid, tartaric acid and an acid salt of an amino acid.

An alternative or a supplement to the use of pH-lowering agents is the use of protease inhibitors, in particular inhibitors of intestinal proteases. The following Table 3 illustrates some of the known intestinal proteases.

TABLE 3
Intestinal Proteases and
their Specific Targets

| | | | |
|---|---|---|---|
| | | pH | |
| PROTEASE | TARGET SITE | OPTIMUM | REMARKS |

| | | | |
|---|---|---|---|
| Trypsin | Lys-Xaa | 8 | |
| | Arg-Xaa | | |
| Chymotrypsin | Tyr-Xaa | 7.0-9.0 | |
| | Phe-Xaa | | |
| | Trp-Xaa | | |
| Elastase | Ala-Xaa | 8.8 | |
| | Val-Xaa | | |
| | Leu-Xaa | | |
| | Ile-Xaa | | |
| | Gly-Xaa | | |
| | Ser-Xaa | | |
| Kallikrein | Arg-Xaa | 7.0-8.0 | |
| | Phe-Arg-Xaa | | preferred |
| | Leu-Arg-Xaa | | preferred |
| Carboxy- | Xaa-Xaa | 7.0-9.0 | from C-terminal |
| peptidase | | | |

Other Optional Ingredients—the Absorption Enhancer

When used, the absorption enhancers are preferably present in a quantity that constitutes from 0.1 to 20.0 percent by weight, relative to the overall weight of the pharmaceutical composition (exclusive of the enteric coating). Preferred absorption enhancers are surface active agents which act both as solubility enhancers and uptake enhancers. Generically speaking, “solubility enhancers” improve the ability of the components of the invention to be solubilized in either the aqueous environment into which they are originally released or into the lipophilic environment of the mucous layer lining the intestinal walls, or both. “Transport (uptake) enhancers” (which are frequently the same surface active agents used as solubility enhancers) are those which facilitate the ease by which peptide agents cross the intestinal wall.

One or more absorption enhancers may perform one function only (e.g., solubility), or one or more absorption enhancers may perform the other function only (e.g., uptake), within the scope of the invention. It is also possible to have a mixture of several compounds some of which provide improved solubility, some of which provide improved uptake and/or some of which perform both. Without intending to be bound by theory, it is believed that uptake enhancers may act by (1) increasing disorder of the hydrophobic region of the membrane exterior of intestinal cells, allowing for increased transcellular transport; or (2) leaching membrane proteins resulting in increased transcellular transport; or (3) widening pore radius between cells for increased paracellular transport.

Surface active agents are believed to be useful both as solubility enhancers and as uptake enhancers. For example, detergents are useful in (1) solubilizing all of the active components quickly into the aqueous environment where they are originally released, (2) enhancing lipophilicity of the components of the invention, especially the peptide active agent, aiding its passage into and through the intestinal mucus, (3) enhancing the ability of the normally polar peptide active agent to cross the epithelial barrier of the brush border membrane; and (4) increasing transcellular or paracellular transport as described above.

When surface active agents are used as the absorption enhancers, it is preferred that they be free flowing powders for facilitating the mixing and loading of capsules during the manufacturing process. Because of inherent characteristics of the present PTH analogs (e.g., their isoelectric point, molecular weight, amino acid composition, etc.) certain surface active agents may interact best with certain peptides. It is preferred, when trying to increase the bioavailability of the present PTH analogs that any surface active agent used as an absorption enhancer be selected from the group consisting of (i) anionic surface active agents that are cholesterol derivatives (e.g., bile acids), (ii) cationic surface agents (e.g., acyl carnitines, phospholipids and the like), (iii) non-ionic surface active agents, and (iv) mixtures of anionic surface active agents (especially those having linear hydrocarbon regions) together with negative charge neutralizers. Negative charge neutralizers include but are not limited to acyl carnitines, cetyl pyridinium chloride, and the like. It is also preferred that the absorption enhancer be soluble at acid pH, particularly in the 3.0 to 5.0 range.

A particularly preferred combination is an acid soluble bile acid together with a cationic surface active agent. An acyl carnitine and sucrose ester is a good combination. When a particular absorption enhancer is used alone, it is preferred that it be a cationic surface active agent. Acyl carnitines (e.g., lauroyl carnitine), phospholipids and bile acids are particularly good absorption enhancers, especially acyl carnitine. Anionic surfactants that are cholesterol derivatives are also used in some embodiments. It is the intent of these preferences to avoid interactions with the peptide agent that interfere with absorption of peptide agent into the blood.

To reduce the likelihood of side effects, preferred detergents, when used as the absorption enhancers of the invention, are either biodegradable or reabsorbable (e.g., biologically recyclable compounds such as bile acids, phospholipids, and/or acyl carnitines), preferably biodegradable. Acylcarnitines are believed particularly useful in enhancing paracellular transport. When a bile acid (or another anionic detergent lacking linear hydrocarbons) is used in combination with a cationic detergent, peptides are believed to be better transported both to and through the intestinal wall.

Preferred absorption enhancers include: (a) salicylates such as sodium salicylate, 3-methoxysalicylate, 5-methoxysalicylate and homovanilate; (b) bile acids such as taurocholic, tauorodeoxycholic, deoxycholic, cholic, glycholic, lithocholate, chenodeoxycholic, ursodeoxycholic, ursocholic, dehydrocholic, fusidic, etc.; (c) non-ionic surfactants such as polyoxyethylene ethers (e.g., Brij 36T, Brij 52, Brij 56, Brij 76, Brij 96, Texaphor A6, Texaphor A14, Texaphor A60 etc.), p-t-octyl phenol polyoxyethylenes (Triton X-45, Triton X-100, Triton X-114, Triton X-305 etc.) nonylphenoxypoloxyethylenes (e.g., Igepal CO series), polyoxyethylene sorbitan esters (e.g., Tween-20, Tween-80 etc.); (d) anionic surfactants such as dioctyl sodium sulfosuccinate; (e) lyso-phospholipids such as lysolecithin and lysophosphatidylethanolamine; (f) acylcarnitines, acylcholines and acyl amino acids such as lauroylcarnitine, myristoylcarnitine, palmitoylcarnitine, lauroylcholine, myristoylcholine, palmitoylcholine, hexadecyllysine, N-acylphenylalanine, N-acylglycine etc.; g) water soluble phospholipids; (h) medium-chain glycerides which are mixtures of mono-, di- and triglycerides containing medium-chain-length fatty acids (caprylic, capric and lauric acids); (i) ethylene-diaminetetraacetic acid; (j) cationic surfactants such as cetylpyridinium chloride; (k) fatty acid derivatives of polyethylene glycol such as Labrasol, Labrafac, etc.; and (l) alkylsaccharides such as lauryl maltoside, lauroyl sucrose, myristoyl sucrose, palmitoyl sucrose, etc.

In some preferred embodiments, and without intending to be bound by theory, cationic ion exchange agents (e.g., detergents) are included to provide solubility enhancement by another possible mechanism. In particular, they may prevent the binding of the present PTH analogs to mucus. Preferred cationic ion exchange agents include protamine chloride or any other polycation.

It is preferred that a water-soluble barrier separate the protease inhibitors and/or the pH-lowering agent from the acid resistant protective vehicle. A conventional pharmaceutical capsule can be used for the purpose of providing this barrier. Many water soluble barriers are known in the art and include, but are not limited to, hydroxypropyl methylcellulose and conventional pharmaceutical gelatins.

In some preferred embodiments, another peptide (such as albumin, casein, soy protein, other animal or vegetable proteins and the like) is included to reduce non-specific adsorption (e.g., binding of peptide to the intestinal mucus barrier) thereby lowering the necessary concentration of the expensive PTH analog active agent. When added, the peptide is preferably from 1.0 to 10.0 percent by weight relative to the weight of the overall pharmaceutical composition (excluding protective vehicle). Preferably, this second peptide is not physiologically active and is most preferably a food peptide such as soy bean peptide or the like. Without intending to be bound by theory, this second peptide may also increase bioavailability by acting as a protease scavenger that desirably competes with the peptide active agent for protease interaction. The second peptide may also aid the active compound's passage through the liver.

All pharmaceutical compositions of the invention may optionally also include common pharmaceutical diluents, glidents, lubricants, gelatin capsules, preservatives, colorants and the like in their usual known sizes and amounts.

The Protective Vehicle

Any carrier or vehicle that protects the PTH analog from stomach proteases and then dissolves so that the other ingredients of the invention may be released in the intestine is suitable. Many such enteric coatings are known in the art, and are useful in accordance with the invention. Examples include cellulose acetate phthalate, hydroxypropyl methylethylcellulose succinate, hydroxypropyl methylcellulose phthalate, carboxyl methylethylcellulose and methacrylic acid-methyl methacrylate copolymer. In some embodiments, the active PTH analog, absorption enhancers such as solubility and/or uptake enhancer(s), and pH-lowering compound(s), are included in a sufficiently viscous protective syrup to permit protected passage of the components of the invention through the stomach.

Suitable enteric coatings for protecting the peptide agent from stomach proteases may be applied, for example, to capsules after the remaining components of the invention have been loaded within the capsule. In other embodiments, enteric coating is coated on the outside of a tablet or coated on the outer surface of particles of active components which are then pressed into tablet form, or loaded into a capsule, which is itself preferably coated with an enteric coating.

It is very desirable that all components of the invention be released from the carrier or vehicle, and solubilized in the intestinal environment as simultaneously as possible. It is preferred that the vehicle or carrier release the active components in the small intestine where uptake enhancers that increase transcellular or paracellular transport are less likely to cause undesirable side effects than if the same uptake enhancers were later released in the colon. It is emphasized, however, that the present invention is believed effective in the colon as well as in the small intestine. Numerous vehicles or carriers, in addition to the ones discussed above, are known in the art. It is desirable (especially in optimizing how simultaneously the components of the invention are released) to keep the amount of enteric coating low. Preferably, the enteric coating adds no more than 30% to the weight of the remainder of pharmaceutical composition (the “remainder” being the pharmaceutical composition exclusive of enteric coating itself). More preferably, it adds less than 20%, especially from 12% to 20% to the weight of the uncoated composition. The enteric coating preferably should be sufficient to prevent breakdown of the pharmaceutical composition of the invention in 0.1N HCl for at least two hours, then capable of permitting complete release of all contents of the pharmaceutical composition within thirty minutes after pH is increased to 6.3 in a dissolution bath in which said composition is rotating at 100 revolutions per minute.

Other Preferences

It is preferred that the weight ratio of pH-lowering agent(s)and/or protease inhibitors to absorption enhancer(s), when present, be between 3:1 and 20:1, preferably 4:1-12:1, and most preferably 5:1-10:1. The total weight of all pH-lowering agents and/or protease inhibitors and the total weight of all absorption enhancers in a given pharmaceutical composition is included in the foregoing preferred ratios. For example, if a pharmaceutical composition includes two pH-lowering agents and three absorption enhancers, the foregoing ratios will be computed on the total combined weight of both pH-lowering agents and the total combined weight of all three absorption enhancers.

It is preferred that the pH-lowering agent and/or protease inhibitor, the PTH analog active agent and the absorption enhancer, when present, (whether single compounds or a plurality of compounds in each category) be uniformly dispersed in the pharmaceutical composition. In one embodiment, the pharmaceutical composition comprises granules that include a pharmaceutical binder having the PTH analog active agent, the pH-lowering agent and the absorption enhancer uniformly dispersed within said binder. Preferred granules may also consist of an acid core, surrounded by a uniform layer of organic acid, a layer of enhancer and a layer of active agent that is surrounded by an outer layer of organic acid. Granules may be prepared from an aqueous mixture consisting of pharmaceutical binders such as polyvinyl pyrrolidone or hydroxypropyl methylcellulose, together with the pH-lowering agents, absorption enhancers and peptide active agents of the invention.

Manufacturing Process

One preferred pharmaceutical composition of the invention includes a size OO gelatin capsule filled with PTH analog, granular citric acid (available for example from Archer Daniels Midland Corp.), taurodeoxycholic acid (available for example from SIGMA), and lauroyl carnitine (SIGMA).

All of the ingredients are preferably selected for eventual insertion into the gelatin capsule, and are preferably powders which may be added to a blender in any order. Thereafter, the blender is run for about three minutes until the powders are thoroughly intermixed. Then the mixed powders are loaded into the large end of the gelatine capsules. The other end of the capsule is then added, and the capsule snapped shut. 500 or more such capsules may be added to a coating device (e.g., Vector LDCS 20/30 Laboratory Development Coating System (available from Vector Corp., Marion, Iowa)).

An enteric coating solution is made as follows. Weigh 500 grams of EUDRAGIT L30 D-55 (a methacrylic acid copolymer with methacylic acid methyl ester, an enteric coating available from ROHM Tech Inc., Maidan, Mass.). Add 411 grams distilled water, 15 grams triethyl citrate and 38 grams talc. This amount of coating will be sufficient to coat about 500 size OO capsules.

The capsules are weighed and placed into the drum of the coating machine. The machine is turned on to rotate the drum (now containing capsules) at 24-28 rpm. The temperature of inlet sprayer is preferably about 45° C. Exhaust temperatures are preferably about 30° C. Uncoated capsule temperature is preferably about 25° C. Air flow is about 38 cubic feet per minute.

A tube from the machine is then inserted into the coating solution prepared as discussed above. The pump is then turned on for feeding solution into the coating device. Coating then proceeds automatically. The machine can be stopped at any time to weigh capsules to determine if the coating amount is sufficient. Usually coating is allowed to proceed for 60 minutes. The pump is then turned off for about five minutes while the machine is still running to help dry the coated capsules. The machine can then be turned off. The capsule coating is then complete, although it is recommended that the capsules be air dried for about two days.

Treatment of Patients

For treatment of osteoporosis, periodic administration is recommended. The attending physician may monitor patient response, PTH analog blood levels, or surrogate markers of bone disease (such as urinary pyridinoline or deoxypyridinoline), especially during the initial phase of treatment (1-6 months). He may then alter the dosage somewhat to account for individual patient metabolism and response.

It is preferred that serum PTH analog peak between 10 and 500 picograms per milliliter, more preferably between 100 and 200 picograms per milliliter, most preferably about 150 picograms per milliliter. The serum levels may be measured by radioimmunoassay techniques known in the art. To achieve such serum concentrations, administration of PTH analog by injection is preferred as a single dosage per day with each dosage containing from about 10 to about 30 micrograms, most preferably about 20 micrograms of PTH analog. However, the most preferred mode of administration is orally once a day with each oral dosage containing from about 0.5 mg to about 20 mg, more preferably from about 1 to about 10 mg of PTH analog.

It is preferred that a single capsule be used at each administration because a single capsule best provides simultaneous release of the PTH analog, pH-lowering agent and absorption enhancers. This is highly desirable because the acid is best able to reduce undesirable proteolytic attack on the polypeptide when the acid is released in close time proximity to release of the polypeptide. Near simultaneous release is best achieved by administering all components of the invention as a single pill or capsule. However, the invention also includes, for example, dividing the required amount of acid and enhancers, when used, among two or more capsules which may be administered together such that they together provide the necessary amount of all ingredients. “Pharmaceutical composition,” as used herein includes a complete dosage appropriate to a particular administration to a human patient regardless of how it is subdivided so long as it is for substantially simultaneous administration.

### EXAMPLE 1

### Efficacy Testing of Various PTH Analogs in Ovariectomized Rats

To compare their effects in bone, amidated fragments of human parathyroid hormone (PTH) were evaluated in the ovariectomized rat model. Aged female Sprague Dawley rats were subject to bilateral ovariectomy or sham surgery. The animals were held untreated for a period of 8 weeks to allow the development of osteopenia. At that point, the ovariectomized animals were segregated into groups, and each group received daily subcutaneous injection of either vehicle or one of the following PTH fragments: PTH[1-30]NH₂, PTH[1-31]NH₂, PTH[1-32]NH₂, PTH[1-33]NH₂, and PTH[1-34]NH₂ at a dose of 9.7 nmol/Kg. Treatment continued for a period of twelve weeks. Primary measures of analyses were 1) bone mineral density (BMD) of the spine (L3-L6) as determined by Dual Energy X-Ray Absorptiometry (DXA), 2) trabecular BMD of the proximal tibia as determined by peripheral Quantitative Computed Tomography (pQCT)and 3) evaluation of cortical bone in the distal tibia by PQCT. Secondary measures included urinary deoxypyridinoline (bone resorption marker), plasma osteocalcin (bone formation marker), histomorphometry of trabecular bone from the fourth lumbar vertebra (L4) and cortical bone from the distal tibia, microcomputed tomography (μCT), and bone strength tests of the spine and femur. Bone strength analysis was done on the femoral midshaft and a lumbar vertebral body.

Bone mineral density (BMD) of the lumbar spine (L3-L6) was assessed throughout the study using Dual Energy X-ray Absorptiometry (DXA). Treatment with the fragments PTH[1-31]NH2, PTH[1-32]NH2, PTH[1-33]NH2, or PTH[1-34]NH2 resulted in a significant increase in lumbar BMD, ranging from 28% to 31% relative to the vehicle-treated ovariectomized animals. There was no significant difference in this parameter among these fragments. However, treatment with PTH[1-30]NH₂ resulted in mean increase in BMD of 13% relative to the vehicle control. This was a significantly smaller increase than that observed with any of the other fragments tested.

To assess the biomechanical consequence of the observed changes in bone mass, compression testing of a lumbar vertebral body was carried out. Treatment with the fragments PTH[1-31]NH₂, PTH[1-32]NH₂, PTH[1-33]NH₂, or PTH[1-34]NH₂ resulted in increases in the maximal load ranging from 100%-121% relative to vehicle-treated ovariectomized animals. There was no significant difference in maximal load among these fragments. Treatment with PTH[1-30]NH₂ resulted in a mean increase in maximal load of 53% relative to the vehicle control group. This was a significantly smaller increase than that observed with any of the other fragments tested.

In other experiments involving an ovariectomized rat model for osteoporosis, good efficacy is shown for the pharmaceutical agents of the invention. For example, the groups used in the study were: Sham, OVX (ovariectomized rats), OVX+PTH(1-30)NH₂, OVX+PTH(1-31)NH₂, OVX +PTH(1-32)NH₂, OVX +PTH(1-33)NH₂ and OVX +PTH(1-34)NH₂.

The peripheral Quantitative Computed Tomography (pQCT)data from the proximal tibia trabecular bone mineral density (BMD) were similar to that for the lumbar spine. As in the lumbar spine, the smallest fragment was different from all the other treatment groups. The remaining groups were not significantly different from each other at Week 20. However, in the proximal tibia at Week 20, the BMD value for trabecular bone of the PTH(1-33)NH₂ group did not continue to increase as did the (1-31), (1-32) and (1-34) groups.

There was no difference between any treatment group and the OVX control in urinary deoxypyridinoline. However, a significant increase in osteocalcin was observed for all PTH fragment treated groups except for PTH(1-30)NH₂. PTH amide fragment 1-34 provided significantly more increase than PTH amide fragments (1-31) and (1-32).

Indices of cortical bone strength derived from PQCT show a general increase with peptides from (1-31) to (1-34) and no effect of hPTH(1-30)NH₂. Trabecular number and thickness were both significantly increased and trabecular separation was decreased as PTH amide fragments (1-31) through (1-34) outperform the PTH(1-30) amide fragment in each respective parameter. The connectivity density of the trabecular bone in the proximal tibia was greatly decreased as a result of OVX. A small increase in connectivity was observed with each peptide. These were only significant for treatment with hPTH(1-30)NH₂ and hPTH(1-31)NH₂.

Furthermore, all PTH amide fragment groups showed a significant decrease in marrow area due to the anabolic action on the endosteal surface. Total area, however, did not increase significantly except hPTH amide (1-31).

In dynamic measurements of bone formation rate/bone surface (BFR/BS), three hPTH amide fragments, hPTH(1-31), (1-33) and (1-34), increased BFR/BS when compared to OVX on the periosteal surface. PTH amide fragments (1-31), (1-32), (1-33) and (1-34) increased cortical bone mass by stimulating bone formation on both the periosteal and endosteal bone surfaces. However, hPTH(1-30)NH₂ fragment's anabolic effect is weak. When examining the percent porosity of the intracortex, all hPTH amide treatment groups were significantly increased when compared to OVX except hPTH(1-32) amide. The OVX control groups' intracortical porosity was significantly increased over that of sham. In the treatment groups, although the cortex is bigger, it is more porous than even the non-treated OVX group.

With regard to the trabecular area, all hPTH amide fragments increased trabecular bone mass in contrast to OVX except for the hPTH amide fragment (1-30). Overall, percent trabecular area, bone volume/total volume, trabecular thickness and number from the micro-CT and histomorphometry agree with each other in showing that the hPTH(1-30) amide fragment is less anabolically effective than the other fragments. The dynamic measure of bone formation rate/bone surface again displays the anabolic effects of all hPTH amide fragments except hPTH(1-30).

Also, in the histomorphometry of LV4, the percent eroded perimeter shows no effect on resorption versus OVX, whereas the percent osteoid perimeter shows an increase in unmineralized bone. This, together with the increased mineralization, all indicates an increase in formation. Generally in the LV4, the PTH amide fragments are not as effective in their anabolic action as they are in the cortex of the tibia-fibula junction. This is also true of the hPTH (1-30) amide fragment.

Cortical analysis of the midshaft of the femur by PQCT shows all fragments to be effective except hPTH(1-30)NH₂. Maximum load by three-point bending of the femur shows all fragments increasing this measurement except for the shortest fragment.

In conclusion, the purpose of the current study was to use the ovariectomized rat model to directly compare the effects of amidated fragments of human PTH ranging in size from 30 to 34 amino acids with respect to bone building effects in trabecular and cortical bone. Accordingly, when tested at a common dose, amidated fragments of human parathyroid hormone ranging in length from [1-31] to [1-34] have a similar positive effect on bone mass and strength in ovariectomized rats. PTH [1-30]NH₂ caused a gain in bone mass that was of a smaller magnitude than the other fragments. Accordingly, all of these truncates tested in this model were efficacious at strengthening bone, particularly human PTH[1-31]NH₂, PTH[1-32]NH₂, PTH[1-33]NH₂, and PTH[1-34]NH₂. Therefore, these four amidated fragments have potential as active agents to treat osteoporosis and bone fracture.

Although the present invention has been described in relation to particular embodiments thereof, many other variations and modifications and other uses will become apparent to those skilled in the art. The present invention therefore is not limited by the specific disclosure herein, but only by the claims.

1. A C-terminal amidated human parathyroid hormone analog PTH 1-32-NH2 (C-terminal amidated SEQ ID No: 20).

2. A C-terminal amidated human parathyroid hormone analog PTH 1-33-NH2 (C-terminal amidated SEQ ID No: 21).

3. A pharmaceutical composition comprising a pharmaceutically effective amount of a C-terminal amidated human parathyroid hormone analog selected from the group of a C-terminal amidated SEQ ID No 20 and a C-terminal amidated SEQ ID No 21, and a pharmaceutically acceptable carrier.

4. The pharmaceutical composition of claim 3, wherein said a C-terminal amidated human parathyroid hormone analog is C-terminal amidated SEQ ID No 20.

5. The pharmaceutical composition of claim 3, wherein said a C-terminal amidated human parathyroid hormone analog is C-terminal amidated SEQ ID No 21.

6. The pharmaceutical composition of claim 3, wherein said composition is suitable for oral delivery.

7. The pharmaceutical composition of claim 6 further comprising at least one agent selected from the group of a pharmaceutically acceptable pH-lowering agent, a protease inhibitor, and a combination thereof.

8. The pharmaceutical composition of claim 7 further comprising an acid resistant protective vehicle effective to transport said pharmaceutical composition through the stomach of a patient while preventing contact between said active peptide agent and stomach proteases.

9. The pharmaceutical composition of claim 7, wherein said pH-lowering agent is present in said pharmaceutical composition in a quantity which, if said composition were added to ten milliliters of 0.1M aqueous sodium bicarbonate solution, would be sufficient to lower the pH of said solution to no higher than 5.5.

10. The pharmaceutical composition of claim 7, wherein said pH-lowering agent is present in a quantity which, if said composition were added to ten milliliters of 0.1M aqueous sodium bicarbonate solution, would be sufficient to lower the pH of said solution to no higher than 3.5.

11. The pharmaceutical composition of claim 7, wherein said protease inhibitor is a stomach and/or intestine protease inhibitor.

12. The pharmaceutical composition of claim 7, wherein said protease inhibitor inhibits an enzyme selected from the group consisting of pepsin, trypsin, chymotrypsin, elastase, kallikrein and carboxypeptidase.

13. The pharmaceutical composition of claim 3, wherein said C-terminal amidated human parathyroid hormone analog is linked to a membrane translocator which is capable of being at least partially cleaved in vivo by an enzyme.

14. The pharmaceutical composition of claim 8, wherein said protective vehicle is present at a weight which is no more than 30% of the weight of the remainder of said pharmaceutical composition.

15. The pharmaceutical composition of claim 8, wherein said protective vehicle is present at a weight which is no more than 20% of the weight of the remainder of said pharmaceutical composition.

16. The pharmaceutical composition of claim 8, wherein said protective vehicle is present at a weight which is between 10% and 20% of the weight of the remainder of said pharmaceutical composition.

17. The pharmaceutical composition of claim 8, wherein said protective vehicle is sufficient to prevent breakdown of said pharmaceutical composition in 0.1N HCl for at least two hours, yet permits complete release of all contents of said pharmaceutical composition within 45 minutes after pH is increased to 6.3 in a dissolution bath in which said composition is rotating at 100 revolutions per minute.

18. The pharmaceutical composition of claim 8 further containing at least one absorption enhancer effective to promote bioavailability of said active agent.

19. The pharmaceutical composition of claim 18, wherein said absorption enhancer is a surface active agent.

20. The pharmaceutical composition of claim 19, wherein said surface active agent is absorbable or biodegradable.

21. The pharmaceutical composition of claim 19, wherein said surface active agent is selected from the group consisting of acylcarnitines, phospholipids and bile acids.

22. The pharmaceutical composition of claim 19, wherein said enhancer is an acyl carnitine.

23. The pharmaceutical composition of claim 22, further including a sucrose ester.

24. The pharmaceutical composition of claim 18, wherein said absorption enhancer is a surface active agent selected from the group consisting of (i) an anionic agent that is a cholesterol derivative, (ii) a mixture of a negative charge neutralizer and an anionic surface active agent, (iii) non-ionic surface active agents, and (iv) cationic surface active agents.

25. The pharmaceutical composition of claim 18, wherein said absorption enhancer is selected from the group consisting of a cationic surfactant and an anionic surfactant that is a cholesterol derivative.

26. The pharmaceutical composition of claim 18, wherein said pharmaceutical composition includes at least two absorption enhancers, one of which is a cationic surface active agent, and another of which is an anionic surface active agent that is a cholesterol derivative.

27. The pharmaceutical composition of claim 26, wherein said anionic surface active agent is an acid-soluble bile acid.

28. The pharmaceutical composition of claim 3, further comprising an amount of a peptide that is not a physiologically active peptide effective to enhance bioavailability of said parathyroid hormone analog.

29. The pharmaceutical composition of claim 7, further comprising a water soluble barrier that separates said pH-lowering agent from said protective vehicle.

30. The pharmaceutical composition of claim 7, wherein said composition includes at least one pH-lowering agent that has a pKa no higher than 4.2.

31. The pharmaceutical composition of claim 7, wherein at least one pH-lowering agent has a solubility in water of at least 30 grams per 100 milliliters of water at room temperature.

32. The pharmaceutical composition of claim 8, wherein all ingredients other than said protective vehicle are uniformly dispersed.

33. The pharmaceutical composition of claim 32, wherein said pharmaceutical composition comprises granules containing a pharmaceutical binder and, uniformly dispersed in said binder, said pH-lowering agent, said absorption enhancer and said peptide active agent.

34. The pharmaceutical composition of claim 18, wherein said composition is a solid dosage form wherein a weight ratio of said pH-lowering agent to said absorption enhancer is between 3:1 and 20:1.

35. The pharmaceutical composition of claim 18, wherein said composition is a solid dosage form wherein the weight ratio of said pH-lowering agent to said absorption enhancer is between 5:1 and 10:1.

36. The pharmaceutical composition of claim 7, wherein said pH-lowering agent is selected from the group consisting of citric acid, tartaric acid and an acid salt of an amino acid.

37. The pharmaceutical composition of claim 7, wherein said pH-lowering agent is present in an amount not less than 300 milligrams.

38. The pharmaceutical composition of claim 37, wherein said pH-lowering agent is present in an amount which is not less than 400 milligrams.

39. The pharmaceutical composition of claim 8, wherein said protective vehicle is a viscous protective syrup.

40. The pharmaceutical composition of claim 36, wherein a water soluble barrier separates said pH-lowering agent from said protective vehicle.

41. A method for preventing or treating osteoporosis comprising administering to a subject in need thereof a pharmaceutically effective amount of a C-terminal amidated human parathyroid hormone analog selected from the group of a C-terminal amidated SEQ ID No 20 and a C-terminal amidated SEQ ID No 21.

42. The method of claim 41, wherein said administration is oral.

43. The method of claim 42, wherein said C-terminal amidated human parathyroid hormone analog is selectively released together with at least one agent selected from the group of a pH-lowering agent, a protease inhibitor, and a combination thereof into a patient's intestine following passage of said analog, pH-lowering agent and/or protease inhibitor through said patient's mouth and stomach under protection of an acid resistant protective vehicle which substantially prevents contact between stomach proteases and said analog.

44. The method of claim 43, wherein the release of said peptide active agent into a patient's intestine is carried out in the presence of at least one absorption enhancer effective to promote bioavailability of said peptide active agent.

45. A method for accelerating the healing of a broken bone comprising administering to a subject in need thereof a pharmaceutically effective amount of a C-terminal amindated human parathyroid hormone analog selected from the group of a C-terminal amidated SEQ ID No 20 and a C-terminal amidated SEQ ID No 21.

46. The method of claim 45, wherein said administration is oral.

47. The method of claim 46, wherein said C-terminal amidated human parathyroid hormone analog is selectively released into a patient's intestine together with at least one agent selected from the group of a pH-lowering agent, a protease inhibitor, and a combination of the foregoing following passage of all orally administered ingredients through said patient's mouth and stomach under protection of an acid resistant protective vehicle which substantially prevents contact between stomach proteases and said analog.

48. The method of claim 47, wherein the release of said peptide active agent into a patient's intestine is carried out in the presence of at least one absorption enhancer effective to promote bioavailability of said peptide active agent.

49. A pharmaceutical composition for oral delivery of a physiologically active peptide agent selected from the group of a C-terminal amidated SEQ ID No 20 and a C-terminal amidated SEQ ID No 21 comprising:
(a) a therapeutically effective amount of said active peptide;
(b) at least one pharmaceutically acceptable pH-lowering agent;
(c) at least one absorption enhancer effective to promote bioavailability of said active agent; and
(d) an acid resistant protective vehicle effective to transport said pharmaceutical composition through the stomach of a patient while preventing contact between said active peptide active agent and stomach proteases;
wherein said pH-lowering agent is present in said pharmaceutical composition in s quantity which, if said composition were added to ten milliliters of 0.1M aqueous sodium bicarbonate solution, would be sufficient to lower the pH of said solution to no higher than 5.5.

50. (canceled)
